(19) United States
(12) Patent Application Publication
(10) Pub. No.: US 2010/0216167 A1
(43) Pub. Date: Aug. 26, 2010

(54) METHODS FOR RELIEVING NEUROPATHIC PAIN BY MODULATING ALPHA 1G T-TYPE CALCIUM CHANNELS AND MICE LACKING ALPHA 1G T-TYPE CALCIUM CHANNELS

(75) Inventors: Heesup SHIN, Uiwang-si (KR); Soon Wook CHOI, Seongnam-si (KR); Dae Soo KIM, Seoul (KR); Heung Sik NA, Seoul (KR); June Sun KIM, Seoul (KR)

Correspondence Address: KLARQUIST SPARKMAN, LLP, 121 SW SALMON STREET, SUITE 1600, PORTLAND, OR, 97204, US

(73) Assignee: Korea Institute of Science and Technology

(21) Appl. No.: 12/775,340

(22) Filed: May 6, 2010

Related U.S. Application Data
(62) Division of application No. 11/721,652, filed on Jun. 13, 2007, now abandoned.

(51) Int. Cl. G01N 33/567

(52) U.S. Cl. 435/7.21

(57) ABSTRACT

The present invention relates to a novel use of a transgenic mouse deficient in α1G T-type calcium channel as an animal model for the study of neuropathic diseases, more precisely, a novel use of a transgenic mouse having resistance against neuripathic pain as an animal model for the development of a therapeutic agent and a treatment method for human neuropathic diseases. The transgenic mouse deficient in α1G T-type calcium channel having resistance against neuropathic pain, provided by the present invention, can be effectively used for the development of a therapeutic agent and a treatment method for human neuropathic diseases.

### CROSS REFERENCE TO RELATED APPLICATIONS

This is a divisional of U.S. patent application Ser. No. 11/721,652, filed Jun. 13, 2007, which is a §371 U.S. National Stage of International Application No. PCT/KR2004/003270, filed Dec. 13, 2004, which was published in English under PCT Article 21(2), which is incorporated herein by reference in its entirety.

### TECHNICAL FIELD

The present invention relates to a novel use of a mouse lacking α1G T-type calcium channel as a model for the development of a therapeutic agent and a method for treating of neuropathic disease. More particularly, the present invention relates to a novel use of a transgenic mouse having resistance against stimulus such as neuropathic pain as a model for the development of a therapeutic agent and a method for treating of neuropathic disease.

### BACKGROUND ART

Voltage dependent calcium channel increases calcium content in cells by the activation of neurons (Tsien, R. W., Annu. Rev. Physiol. 45, 341-358, 1983), and is divided into high-voltage dependent channel and low-voltage dependent channel (Tsien, R. W. et al., Trends Neurosci. 18, 52-54, 1995). As a representative low-voltage dependent calcium channel found in human, T-type calcium channel is divided into three classes by genotype for alpha subunit, which are Cav3.1 (α1G), 3.2 (α1H) and 3.3 (α1I) (Perez-Reyes, E., Physiol. Rev. 83, 117-161, 2003). α1G calcium channel is involved in the generation of multiple burst firings of neurons in thalamic nucleus, and major pathological functions of the channel have been recently disclosed (Kim, D. et al., Science 302, 117-119, 2003; Kim, D. et al., Neuron 31, 35-45, 2001).

The pronociceptive role of T-type calcium channel among signal transduction process of pains has been a major target of study on the development of a therapeutic agent for pain relief (Carbone, E. & Lux, H. D., Nature 310, 501-2, 1984; Todorovic, S. M. et al., Neuron 31, 75-85, 2001; Todorovic, S. M. et al., Brain Res. 951, 336-40, 2002; Ikeda, H. et al., Science 299, 1237-40, 2003; Heinke, B., et al., Eur. J. Neurosci. 19, 103-11, 2004).

However, according to a recent report, thalamus has an antinociceptive function, meaning that it hinders pain signal transduction by α1G T-type calcium channel, and in fact, the changed firing pattern of thalamocortical neuron affects the thalamocortical mechanism of inhibiting response to a pain, resulting in hyperalgesia against abdominal pain (Kim, D. et al., Science 302, 117-119, 2003).

The change of plasiticity in synapse between pain reactive afferent and spinal dorsal horn neuron such as spinal cord causes over-activation of central nerve, resulting in pathogenesis of neuropathic pain (Mayer, D. J. et al., Proc. Natl. Acad. Sc.i U.S.A. 96, 7731-6, 1999; Woolf, C. J. & Salter, M. W., Science 288, 1765-9, 2000; Hunt, S. P. & Mantyh, P. W., Nat. Rev. Neurosci. 2, 83-91, 2002).

Recently, Ikeda and his collegues proposed that T-type calcium channel might induce long-term potentiation (LTP) in synapse (Ikeda, H. et al., Science 299, 1237-40, 2003). That is, T-type calcium channel might be involved in the generation and maintenance of neuropathic pain. Blockade of spinal T-type calcium channel by ethosuximide inhibits neuronal response of horn (Matthews, E. A. & Dickenson, A. H., Eur. J. Pharmacol. 415, 141-9, 2001), and the systemic administration of mibefradil or ethosuximide can effectively reverse ethological signal of neuropathic pain (Dogrul, A. et al., Pain 105, 159-68, 2003).

Thus, the present inventors induced spinal nerve ligation (SNL) in mice lacking the gene above, and then investigated the response of the transgenic mice for various abnormal pains caused by such nerve injury in order to investigate the role of α1G T-type calcium channel in pain reactivity and pathological pain.

As a result, it was observed that normal response for general pain and for other stimuli such as physical stimulus, low temperature and high temperature was significantly decreased in the transgenic (knock-out) mice lacking α1G T-type calcium channel, and so the present inventors completed this invention by confirming that pain caused by nerve injury could be relieved by regulating α1G T-type calcium channel.

### DISCLOSURE

### SUMMARY OF THE INVENTION

It is an object of the present invention to provide a use of a mouse deficient in α1G T-type calcium channel as a model for the development of a therapeutic agent and a method for treatment of neuropathic diseases, and a method for relieving pains caused by nerve injury by regulating α1G T-type calcium channel.

### DETAILED DESCRIPTION OF THE INVENTION

In order to achieve the above object, the present invention provides a method for using the transgenic mouse deficient in α1G T-type calcium channel as an animal model for the study on the development of a therapeutic agent and a method for treatment of neuropathic diseases.

The present invention also provides a method for relieving neuropathic pain caused by nerve injury by inhibiting α1G gene encoding a pore forming subunit of α1G T-type calcium channel.

The present invention further provides a screening method for α1G T-type calcium channel inhibitor by using a cell line expressing α1G T-type calcium channel.

Hereinafter, the present invention is described in detail.

The present invention provides a method for using a transgenic mouse deficient in α1G T-type calcium channel as an animal model for the development of a therapeutic agent and a method for treatment of neuropathic diseases.

The present inventors prepared a transgenic mouse whose genotype is α1G−/− by using a fertilized egg (Korean Collection for Type Cultures, Korea Research Institute of Bioscience and Biotechnology, Accession No: KCTC 10086BP) having α1G+/− genotype of T-type calcium channel. Particularly, a fertilized egg whose genotype is α1G+/− was transplanted in a surrogate mouse to prepare a heterozygote mouse whose genotype is α1G+/−. Then, the heterozygote transgenic female and male mice were mated to prepare a homozygote mouse whose genotype is α1G−/−.

T-type calcium channel is sub-divided into α1G, α1H and α1I according to the pore forming subunit. In the present invention, among those three sub-types, α1G protein, a constituent of α1G T-type calcium channel, was inhibited to prepare a transgenic mouse having α1G−/− genotype, and then spinal nerve ligation (SNL) was induced therein for the experiments on response against neuropathic pain caused by nerve injury.

As a result, neuropathic pain caused by nerve injury induced by spinal nerve ligation was significantly decreased in the transgenic mouse deficient in α1G T-type calcium channel, comparing to a wild type mouse (see FIG. 1 and FIG. 2).

Therefore, the transgenic mouse deficient in α1G T-type calcium channel, in which neuropathic pain was induced, can be used as an animal model for the development of a therapeutic agent and a treatment method for neuropathic diseases.

The present invention also provides a method for relieving neuropathic pain by suppressing a gene encoding a pore forming subunit of α1G T-type calcium channel.

The present inventors performed experiments on pain response against various stimuli after inducing spinal nerve ligation in α1G gene knock-out mouse. As a result, the response against neuropathic pain after spinal nerve ligation was remarkably decreased in the transgenic mouse deficient in α1G T-type calcium channel. The result indicates that neuropathic pain can be relieved by suppressing α1G gene in a wild-type individual. That is, the transmission of pain can be hindered by regulating α1G T-type calcium channel by suppressing the function of α1G gene, resulting in relieving neuropathic pain.

The present invention further provides a screening method for an α1G inhibitor by using a cell line expressing α1G T-type calcium channel.

It was proved in the present invention that neuropathic pain could be relieved by suppressing α1G gene. Therefore, any substance that is able to suppress α1G gene can be used as a pain reliever for the treatment of neuropathic diseases. For the screening of such neuropathic pain reliever, it is important to investigate the activity of inhibiting α1G T-type calcium channel of a target substance by using a cell line expressing α1G, which might provide an important clue for the development of a therapeutic agent for neuropathic diseases.

T-type calcium channel is a LVA calcium channel, meaning it is activated under low voltage. Membrane potential of most cells expressing T-type calcium channel is not hyperpolarized enough to activate the T-type calcium channel. Thus, it is necessary to express potassium channel, which contributes greatly to the formation of membrane potential, together with α1G T-type calcium channel in a cell line, in order to activate α1G calcium channel with keeping membrane potential stable by lowering the membrane potential a little toward hyperpolarization.

The present inventors have previously deposited a cell line expressing α1G T-type calcium channel together with potassium channel to activate α1G T-type calcium channel (Accession No: KCTC 10519BP), which enables the screening of an inhibitor for the activation of α1G T-type calcium channel.

Particularly, the method for the screening of an inhibitor suppressing the activity of α1G T-type calcium channel includes following steps:

i) Culturing a cell line expressing α1G;

ii) Treating an inhibitor candidate for the suppression of the activity of α1G T-type calcium channel at different concentrations to the cells cultured in the above step i); and

iii) Measuring calcium current in the cell line treated with the above inhibitor candidate of ii).

At first, a cell line expressing α1G was cultured, and then an inhibitor candidate was treated to the cell culture solution at different concentrations. The inhibition of electric current by α1G T-type calcium channel was measured at each concentrations using voltage-clamp method (Dillon G. H. et al., Mol. Pharmacol. 1993, Bodding M., J. Biol. Chem. 2004). Based on the measurement, a substance inhibiting most effectively the activity of α1G T-type calcium channel and the concentration thereof were determined.

The inhibitor of the activity of α1G T-type calcium channel, confirmed by the screening above, is a prospective candidate for a therapeutic agent for neuropathic diseases.

### BRIEF DESCRIPTION OF DRAWINGS

FIG. 1A is a graph showing that spontaneous pain resulted from nerve injury in a transgenic mouse deficient in α1G T-type calcium channel was significantly decreased after each 14 and 21 days from the inducement of the nerve injury, comparing to a control group:

| | |
|---|---|
| : Wild-type, | ∘: α1G−/−. |

FIG. 1B is a graph showing that mechanical allodynia caused by nerve injury was remarkably decreased in a transgenic mouse deficient in α1G T-type calcium on day 1, day 7 and day 21 after the nerve injury was induced, comparing to a control group:

| | |
|---|---|
| : Wild-type, | ∘: α1G−/−. |

FIG. 1C is a graph showing that cold allodynia (15° C.) caused by nerve injury was significantly decreased on day 21 after the nerve injury was induced in a transgenic mouse deficient in α1G T-type calcium channel, comparing to a control group:

| | |
|---|---|
| : Wild-type, | ∘: α1G−/−. |

FIG. 2A is a graph showing that thermal hyperalgesia (infrared strength 30) caused by nerve injury was significantly decreased in a transgenic mouse deficient in α1G T-type calcium channel on day 1, day 14 and day 21 after the inducement of the nerve injury, comparing to a control group:

| | |
|---|---|
| : Wild-type, | ∘: α1G−/−. |

FIG. 2B is a graph showing that thermal hyperalgesia (infrared strength 60) caused by nerve injury was significantly decreased in a transgenic mouse deficient in α1G T-type calcium channel on day 14 and day 21 after the inducement of the nerve injury, comparing to a control group:

| | |
|---|---|
| : Wild-type, | ∘: α1G−/−. |

### PREFERRED EMBODIMENTS OF THE INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Preparation and Maintanance of α1G−/− Transgenic Mouse

### <1-1> Preparation of α1G−/− Transgenic Mouse

The present inventors prepared a transgenic mouse whose genotype is α1G−/− by using a fertilized egg (Korean Collection for Type Cultures, Korea Research Institute of Bioscience and Biotechnology, Accession No: KCTC 10086BP) whose genotype is α1G+/− of T-type calcium channel. Particularly, a fertilized egg whose genotype is α1G+/− was transplanted in a surrogate mother mouse to prepare a heterozygote mouse whose genotype is α1G+/−. A female and a male heterozygote mouse were mated to prepare a homozygote mouse whose genotype is α1G−/−.

### <1-2> Maintenance of Animals

The transgenic mouse was raised under 12 hour of light and 12 hour of dark cycle, during which water and food were supplied without limitation. The light cycle was started at 6 am. All the behavioral experiments including animal protection and pain tests were conducted by following ethical guidelines proposed by Korea Institute of Science and Technology and Institutional Animal Care and Use Committee affiliated with International association for the Study of Pain.

### Example 2

### Preparation of Surgical Operation for Nerve Injury Induced Mouse: Spinal Nerve Ligation (SNL)

A test animal was anesthetized by gas mixture of oxygen and enflurane (2% for inducement, and 0-5% for maintenance), followed by surgical operation. L5 spinal nerve was ligated by following the method of Kim and Chung (1992). Briefly, spine ranging from L4 to S2 was open and L6 vertebral transverse process was eliminated. L5 spinal nerve was tightly ligated by using 6-0 silk threads under dissecting microscope. After complete stanching, the wound was sutured.

### Example 3

### Analysis of Response Against Stimulus

### <3-1> Spontaneous Pain Test

In order to investigate spontaneous pain, behavorial evaluation method for spontaneous pain that was modified from formalin test system (Dubuisson, 1977) was used. A test animal was given a free hand in a transparent plastic cylinder (6 cm in diameter×16 cm in height) with the top opened. The animal was let adapt to the circumstance for 20 minutes before observation was start. During three-minute observation, cumulative time that the animal was up in the air was recorded. However, the time that the animal lifted up its feet during movement or for back to its place was not measured. An average score for two times experiments was calculated.

### <3-2> Von Frey Filament Test

In order to quantify the mechanical sensitivity of paw, up/down method was used to measure withdrawal threshold of paw against von Frey filament (Chaplan, 1994). In each test, a test animal was put on the metal mesh floor in a transparent plastic chamber (9.5×5.5×5 cm³). 50% withdrawal threshold was measured by using a set of von Frey filament (0.02, 0.07, 0.16, 0.4, 1, 2, 4, 6 g, Stoelting, Wood Dale, Ill., USA). The active paw lift for the adaptation to von Frey was regarded as withdrawal response. The first stimulus was 0.4 g filament. If there was a withdrawal response, the next weak filament was given, but if there was not a withdrawal response, the next strong filament was given. 50% threshold interpolation was performed by the method of Dixon (1980).

### <3-3> Tail Clip Test

A strong mechanical stimulus was given to the tail by using an alligator clip (Fine Science Tools Inc., North Vancouver, Canada). The latent time to response (shaking and biting) was investigated.

### <3-4> Paw Withdrawal Test

The present inventors measured hind-paw withdrawal latency by modified Hargreaves' method. The test was performed at low (IR 30) and high (IR 60) intensities. Cut-off time was set to 15 seconds to prevent tissue damage. Thermal stimulus was given to each paw 4-5 times at 5-10 minutes interval, and the average time for lifting up the paw was measured.

### <3-5> Tail Flick Test

Cut-off time was also set to 15 seconds for the tail flick test to minimize tissue damage. The test was performed at high (IR 50) intensity. Thermal stimulus caused by radiant heat was given to the tail 5 times and then the average latent time was calculated. At least 10 minute-intermission was permitted between each trial.

### <3-6> Hot Plate Test

A mouse was adapted on the metal floor in a transparent test box (15×15×25 cm³) for one hour. Then, the mouse was transferred into a box which was pre-heated to 52.5° C. in a thermal control bath. The latent time to the first licking or jumping was measured.

### <3-7> Cold Sensitivity Test

In order to quantify the cold sensitivity of paws, a drop of cold water (15° C.) was dropped onto the paw and then sudden shrink of the paw was measured. The mouse was put on the metal mesh floor in a transparent plastic chamber, and then had the sole of its hind-paw contacted cold water. To do so, a drop of cold water was formed by using a small polyethylene tube fragment connected to a syringe. The drop of cold water was given to each hind-paw five times (at 5 minutes interval). The frequency of paw withdrawal was calculated as percentage (%) (Frequency of paw withdrawal/total trial number×100).

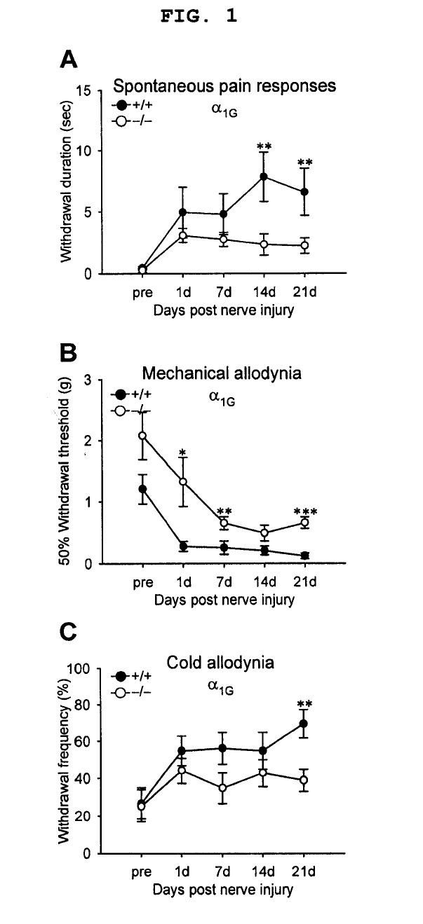

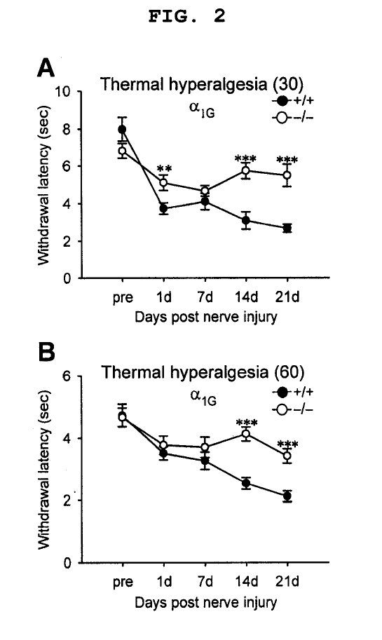

As a result, spontaneous pain response (FIG. 1A), mechanical allodynia (FIG. 1B), cold allodynia (FIG. 1C) and thermal hyperalgesia (FIG. 2) were all observed in both mutant mice having α1G+/+ and α1G−/−, in which spinal nerve ligation (SNL) was induced (Friedman repeated measures analysis of variance with post-hoc test by Dunnett's method, *p<0.05). However, neuropathic pain response was significantly decreased in a transgenic mouse having α1G−/− genotype, comparing to a wild-type mouse.

As shown in FIG. 1A, a transgenic mouse having α1G−/− genotype had shorter continuance of paw withdrawal than a mouse having α1G+/+ genotype, which was proved through spontaneous pain response (Mann-Whitney rank sum test, *p<0.05 [symbol] **p<0.01). In addition, mechanical and cold allodynia was also reduced in the α1G−/− mouse (FIG. 1B and FIG. 1C, Mann-Whitney rank sum test, **p<0.01), and further thermal hyperalgesia was also greatly decreased in the mouse (FIG. 2A and FIG. 2B, Mann-Whitney rank sum test, **p<0.01 and ***p<0.001).

### INDUSTRIAL APPLICABILITY

As explained hereinbefore, the present invention relates to a use of a transgenic mouse deficient in α1G T-type calcium channel having resistance against pain caused by nerve injury as an animal model for the study of human neuropathic pain related diseases. The animal model provided by the present invention can be effectively used for the development of a therapeutic agent and a treatment method for human neuropathic diseases.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A method for identifying a neuropathic pain reliever comprising screening for an inhibitor suppressing the activity of α1G T-type calcium channel by using a cell line expressing α1G T-type calcium channel.

2. The method as set forth in claim 1, wherein the cell line is deposited as KCTC 10519BP.

3. The method as set forth in claim 1, wherein the method comprises:
culturing a cell line expressing α1G;
treating the cells cultured with a compound or composition to be tested;
measuring calcium current in the cell line; and
determining whether the compound or composition inhibits the activity of α1G T-type calcium channel in comparison with a control.

4. The method as set forth in claim 3, wherein the measurement of calcium current is performed by a voltage-clamp method.
